(19) United States
(12) Patent Application Publication
(10) Pub. No.: US 2005/0282268 A1
(43) Pub. Date: Dec. 22, 2005

(54) Culture microscope and computer program controlling culture microscope

(75) Inventor: Akitsugu Kagayama, Hachioji-shi (JP)

Correspondence Address: FRISHAUF, HOLTZ, GOODMAN & CHICK, PC, 220 5TH AVE FL 16, NEW YORK, NY, 10001-7708, US

(73) Assignee: Olympus Corporation, Tokyo (JP)

(21) Appl. No.: 11/137,161

(22) Filed: May 24, 2005

(30) Foreign Application Priority Data
May 26, 2004 (JP) 2004-155897

(51) Int. Cl. C12M 1/34

(52) U.S. Cl. 435/288.7; 382/133; 359/395; 359/398; 435/303.1

(57) ABSTRACT

A culture microscope has an incubator chamber which controls a culture environment in which cells are cultured, an imaging optical system which photographs the cells, and a controller which controls a time-lapse photographing performed by the imaging optical system.

### CROSS-REFERENCE TO RELATED APPLICATIONS

This application is based upon and claims the benefit of priority from the prior Japanese Patent Application No. 2004-155897, filed May 26, 2004, the entire contents of which are incorporated herein by reference.

### BACKGROUND OF THE INVENTION

1. Field of the Invention

The present invention relates to a microscope combined with a culture apparatus. More particularly, the invention relates to a culture microscope which is used to observe and photograph a sample of a living biological system, e.g., a living cell, for a long time.

2. Description of the Related Art

Organisms have high complexity. It is therefore not easy to understand their configurations or functions. In view of this, a simple experimental system is employed, which uses a cultured cell as a minimum unit that can represent life events. Use of a cultured cell can accomplish an experiment in which an analysis of, e.g., responses of hormones, is not affected by other factors in a living body. In other words, the functions of genes can be analyzed by introducing or obstructing genes.

In order to culture cells, an environment simulating the inside of a living body must be used. Therefore, temperature is set to 37° C. corresponding to the body temperature, and a culture medium simulating an intercellular fluid is used. The culture medium used includes a nutritive source such as amino acid as well as buffer carbonate, i.e. sodium hydrogen carbonate for PH adjustment. The buffer carbonate enters into an equilibrium state in the presence of air that contains carbon dioxide having a partial pressure as high as 5%. The buffer carbonate is used for culture in an open system such as a dish. An environment at a high humidity of 95-100% is required to avoid evaporation of fluid from the culture medium.

To culture cells, a carbon dioxide incubator is used, which meets the environmental conditions specified above. A phase-contrast microscope or a differential interference contrast microscope is used to observe the state of the cell. To observe GFP or the manifestation of thereof, or the like, a fluorescent microscope is used. To acquire and display a still or moving picture of the cell, a CCD camera and a controller (i.e., a personal computer) are employed. A culture microscope that is a combination of a CCD camera and a controller has been proposed.

To observe cells being cultured, by using a microscope over a long time or over a long period, a time-lapse scheme is employed. This scheme acquires images in time series. The time-lapse scheme is used to photograph a sample at fixed intervals, thus acquiring images of the sample. Therefore, the scheme makes it easy to determine how the sample, i.e., cell, has changed over a long time. For example, a cell is first photographed with 1 msec exposure time and then at intervals of one hour, for 24 hours. In this case, 25 images of the cell are acquired. If these images are sequentially displayed, how the cell has changed every hour will be confirmed. Photographing intervals become shorter, for example 30 minutes or 15 minutes. Then, the cell moving faster can be observed.

To photograph cells at different positions or to photograph a cell at its different parts, the motorized stage of the microscope used is moved to bring the focal point of the microscope to each part, or to bring the each part of the cell to the focal point. The motorized stage is moved at the photographing intervals descried above. This method of photographing the plurality of cells or parts shall be referred to as “multipoint time-lapse.”

To culture cells for a long time, the culture medium must be replaced with a new one when it becomes degraded. Generally, the operator takes the sample container from the incubator chamber of the culture microscope, removes the medium from the container and sets a new culture medium in the container.

Ordinary multipoint time-lapse including ordinary single-point time-lapse is performed by moving the motorized stage and controlling the microscope, thereby photographing the sample at multiple points for the first time. Immediately before photographing the sample for the second time, the microscope remains not operating or remains in a standby state. If the non-operating time is sufficiently long, the culture medium can be replaced by a new one while the microscope remains in the standby state.

### BRIEF SUMMARY OF THE INVENTION

A culture microscope according to this invention has an incubator chamber in which a culture environment for cells is controlled, an imaging device which photographs a cell to acquire images thereof, and a controller which controls the time-lapse photographing performed by the imaging device.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a conceptual representation of an apparatus according to this invention;

FIG. 2 is a diagram showing the internal structural of the main body of a culture microscope according to the invention;

FIG. 3 is a block diagram of an electrically controllable unit;

FIGS. 4A and 4B illustrate the incubator chamber that has been opened for replacement of the culture medium;

FIGS. 5A, 5B and 5C illustrate the tray removed from the incubator chamber, for replacement of the culture medium;

FIGS. 6A and 6B are flowcharts, or a simplified program that a computer executes to control the culture microscope;

FIG. 7 is a diagram explaining a live image window and a control panel GUI;

FIG. 8 is a diagram explaining a caution dialog;

FIG. 9 is a diagram illustrating a time-lapse schedule window;

FIG. 10 is a view illustrating a dialog which urges an operator to replace a culture medium;

FIG. 11 is a view illustrating a dialog which shows a time remaining until the next photographing;

FIG. 12 is a diagram illustrating a restart dialog;

FIG. 13 is a diagram illustrating a cancel dialog;

FIG. 14 is a diagram illustrating a daily base time-lapse schedule window;

FIG. 15 is a diagram showing an alarm buzzer and an alarm display; and

FIG. 16 is a diagram depicting the structure of a sample container.

### DETAILED DESCRIPTION OF THE INVENTION

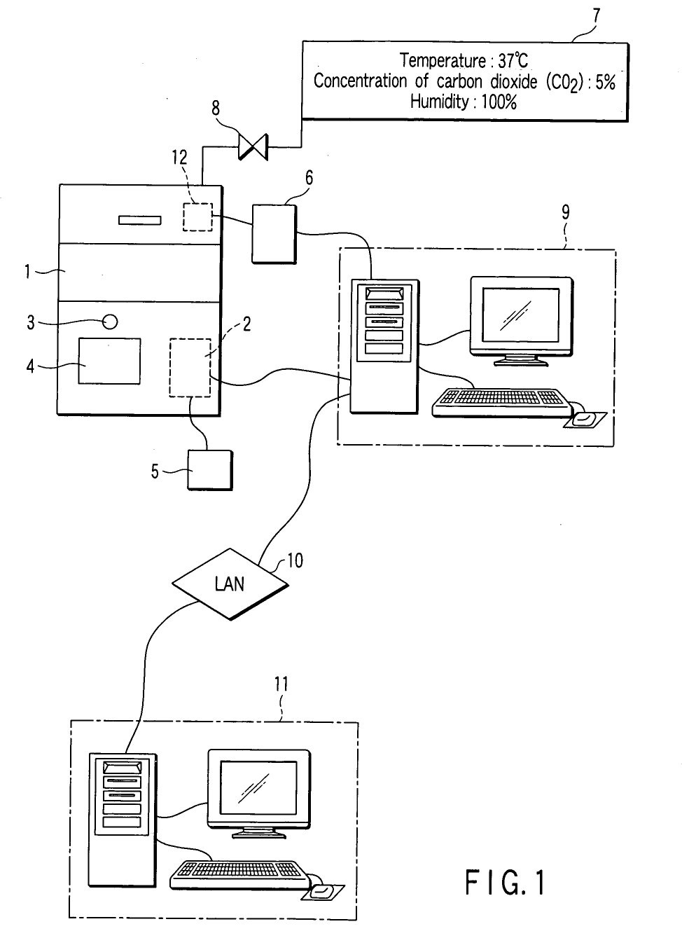

A first embodiment according to the invention will be described, with reference to the accompanying drawings. FIG. 1 is a conceptual representation of an apparatus according to the present invention.

A culture microscope main body 1 contains an incubator chamber, in which cells are cultured. The chamber is integrated with a microscope section that is used to observe a cell. The main body 1 contains a controller 2. The controller 2 controls other component, described later. The controller 2 is arranged in the main body 1, thus reducing the space occupied by the culture microscope. Nonetheless, the controller 2 may be arranged outside the main body 1 if the heat that it generates while operating may influence other components provided in the main body 1. The culture microscope main body 1 further has an alarm buzzer 3 and an alarm display 4. The alarm buzzer 3 generates an alarm when any trouble occurs during the experiment or the operation. The alarm display 4 displays an alarm or an operation instruction when any trouble occurs. Particularly, the alarm display 4 is made of a touch panel 4a that functions as an operation panel, as well. The operator may touch the touch panel 4a to select an operation, in accordance with the instruction displayed by the alarm display 4.

A focus handle/joystick 5 comprising a focus handle and/or a joystick is connected to the controller 2. When manipulated, the focus handle/joystick moves a microscope section (described later) in a Z-axis direction, in which the focal point is moved to or away from the sample. When manipulated, the handle/joystick can move an R stage and a θ stage. The θ stage is a motorized stage that can rotate an axis. The R stage is a motorized stage that can move in the radial direction perpendicular to the axis of the θ stage. The R and θ stages, which are used to reduce the size of the apparatus, may be replaced by an XY stage of ordinary type.

The culture microscope main body 1 has a temperature control heater 12, which is provided in the incubator chamber. The main body 1 has a temperature controller 6, which controls the heater 12.

The controller 2 and the temperature controller 6 are connected to a computer 9 by an interface such as RS-232C. Thus, the computer 9 can control both controllers 2 and 6.

A tank 7 is provided outside the incubator chamber of the culture microscope main body 1. The tank 7 contains mixed air whose temperature of 37° C., humidity of 95 to 100% and carbon dioxide (CO₂) with partial pressure of 5%. The values specified can generally be adjusted. The mixed air is supplied into the tank 7 by opening an electromagnetic valve 8. The mixed air may be replaced with carbon dioxide gas. A water tank, not shown, may be set in the incubator chamber for maintaining the humidity at a specific value in the incubator chamber. Carbon dioxide gas can be supplied into the incubator chamber, without maintaining the temperature in the tank 7 at 37° C. The valve 8 may be controlled by a non-illustrated controller, which in turn is controlled by the computer 9.

The computer 9 is connected to a network 10, such as LAN Internet. The network 10 is connected to a remote computer 11. The computer 9 can be controlled from the remote computer 11 via the network 10. Hence, the remote computer 11 can control the culture microscope main body 1.

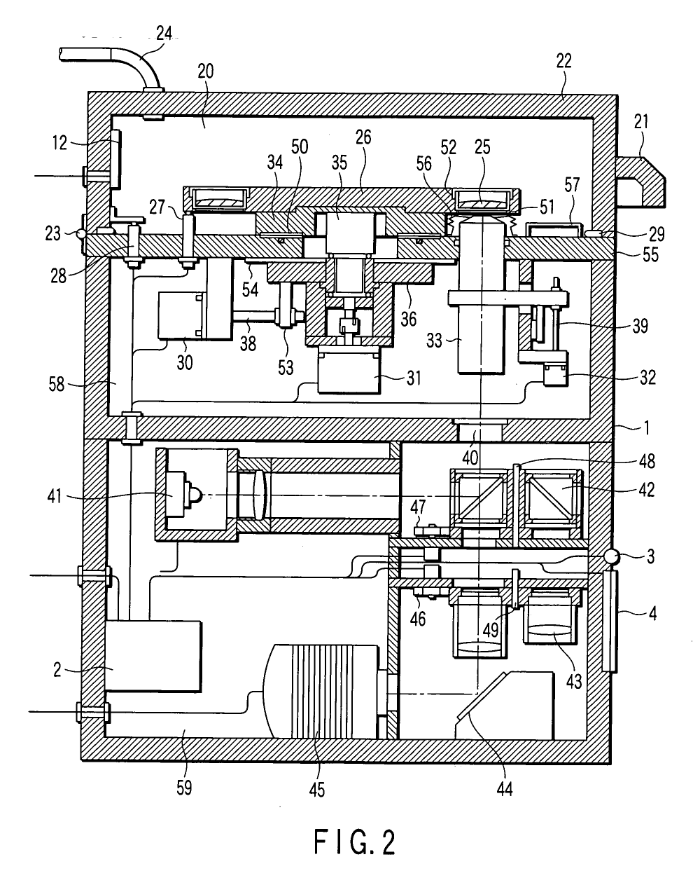

FIG. 2 depicts the internal structural of the culture microscope main body 1.

A lid 22 seals the incubator chamber 20 from the outside. In the chamber 20, the temperature, humidity, and carbon dioxide (CO₂) concentration are maintained at fixed values suitable for a culture environment, or are positively controlled. The mixed air is supplied from the tank 7 through a gas pipe 24. Unnecessary air is discharged through a pipe, not shown. The lid 22 has a knob 21 and is coupled to the chamber 20 with a hinge 23. The operator may hold the knob 21 and open or close the lid 21, rotating the lid 21 around the shaft of the hinge 23. When the lid 22 is opened, a lid open/close sensor 28 detects the movement of the lid 22, informing the controller 2 of the opening of the lid 22.

The heater 12 is provided in the incubator chamber 20. A temperature sensor, not shown, detects the temperature in the chamber 20. When this sensor detects that the temperature has reached a predetermined value, e.g., 37° C., or a smaller value, the heater 12 automatically operates to maintain the temperature. Only one heater 12 is shown in FIG. 2. Nonetheless, other heaters may be attached to the lid 22 or base 55. In this case, the temperature distribution in the chamber 20 is more uniform.

A tray 26 is provided in the incubator chamber 20. The tray 26 has sample-holding holes 52 for holding sample containers 25. The sample container 25 can be moved up from the tray 26. While held in one sample-holding hole 52, each sample container 25 contacts, at bottom, the annular protrusion 51 provided in the hole 52. The protrusion 51 prevents the container 25 from dropping. The sample containers 25 can be positioned with respect to the tray 26. The bottom of each sample container 25 is made of transparent glass or resin. The sample in the container 25 can therefore be observed through an objective lens 33.

Each sample container 25 has a lid 57. After the container 25 is taken out of the incubator chamber 20 for replacement of the culture medium, the sample in the container 25 may be cooled. When the container 25 is put back into the incubator chamber 20, dew drops may be formed on the lid 57. In this case, the lid 57 is replaced by one of spare container lids that are stored in a space provided in the incubator chamber 20. Stored in the chamber 20, each spare container lids will not be cooled. As FIG. 16 shows, each sample container 25 has a member 90, a top 91, a member 92, and a bottom 93. The top 91 and bottom 93 are made of, for example, glass. Thus, through these members 91 and 93, anything inside the container 25 can be observed. The members 90 and 92 are adhered to the top 91 and bottom 93, and can be removed from the top 91 and bottom 93, respectively. The members 90 and 92 are made of material, such as metal, which has large heat capacity. Hence, dew drops are prevented from being formed on the top 91 or the bottom 93.

The tray 26 can be attached and detached to and from a rotary base 34. When the tray 26 is detached, a tray attachment/detachment sensor 27 informs the controller 2 of this event. As FIG. 2 shows, the tray attachment/detachment sensor 27 is a push-button type. Nonetheless, the sensor 27 may be of any other type that can detect that the tray 26 has been detached from the rotary base 34.

The rotary base 34 is attached to a θ rotary shaft 35. Thus, it rotates the tray 26 when a θ stage motor 31 drives the shaft 35.

An R stage motor 30 drives a lead screw 38. A linearly movable base 36 having a nut 53 is thereby moved to the left or the right. The base 36 is guided by a linear guide 54 and can move only in a straight line. The θ rotary shaft 35 is rotatably attached to the linearly movable base 36. When the base 36 moves to the left or the right, the rotary base 34 is moved in the same direction. This structure implements a stage that can move a sample in an Rθ polar coordinate system.

A base 55 partitions the incubator chamber 20 from a motor chamber 58. The chambers 20 and 58 are sealed from each other. Highly humid air does not flow into the incubator chamber 20. A tabular sheet 50 is interposed between the rotary base 34 and the base 55, allowing the rotary base 34 to slide on the base 55.

Bellows 56 surround that part of the objective lens 33, which is exposed to the interior of the incubator chamber 20. The bellows 56 are attached at an end to an end part of the objective lens 23 and to the base 55, by using an adhesive or the like, and is thereby sealed. As a result, highly humid air does not flow into the motor chamber 58 through the gap between the base 55 and the objective lens 33.

The objective lens 33 moves up and down when a Z stage motor 32 turns a lead screw 39. When the objective lens 33 moves up and down, the focal point can be placed on the sample. The bellows 56 can expand and contract even when the objective lens 33 moves up and down. This is because the bellows 56 are formed of soft resin such as rubber. Thus, the bellows 56 remain sealed.

The temperature in the microscope chamber 59 is maintained to prevent expansion the optical members provided in the chamber 59. A heater or the like, not shown, is used to maintain the temperature.

The controller 2 is provided in the microscope chamber 59. Wires are connected to the units that are arranged in the chamber 59. An LED 41 as a light source applies observation light through a window 40, a cube 42 and the objective lens 33, thus illuminating the sample. The light reflected from the sample passes through the objective lens 33, the window 40, the cube 42 and a magnification-changing lens 43. A mirror 49 deflects the light beam by 90°. The light beam is then applied to a CCD camera 45. The mirror 44 is used, providing a space for the CCD camera 45. If an ample space is available for the camera 45, the light beam need not be deflected at all. Fluorescent excitation and observation are also possible such as an ordinary microscope.

The LED 41 may be replaced by a mercury lamp, not shown, and an optical fiber. Any mercury lamp cannot be switched at so high a speed as the LED 41. A shutter must therefore be attached to the lamp and be opened and closed to apply, on and off, the light emitted by the mercury lamp. The controller 2 can control both the mercury lamp and the shutter. A light may enter the CCD camera 45, without being transmitted through the magnification-changing lens 43. That is, the magnification-changing lens 43 may be moved onto and from the optical path extending from the objective lens 33 to the CCD camera 45.

The cube 42 can be rotated about a shaft 48 by a cube turret motor 47 and replaced with a cube having a different wavelength. The cube turret motor 47 is controlled by the controller 2.

The magnification-changing lens 43 can rotate about a shaft 49 by a lens turret motor 46 and can be replaced with a lens having a different magnification. The lens turret motor 46 is controlled by controller 2.

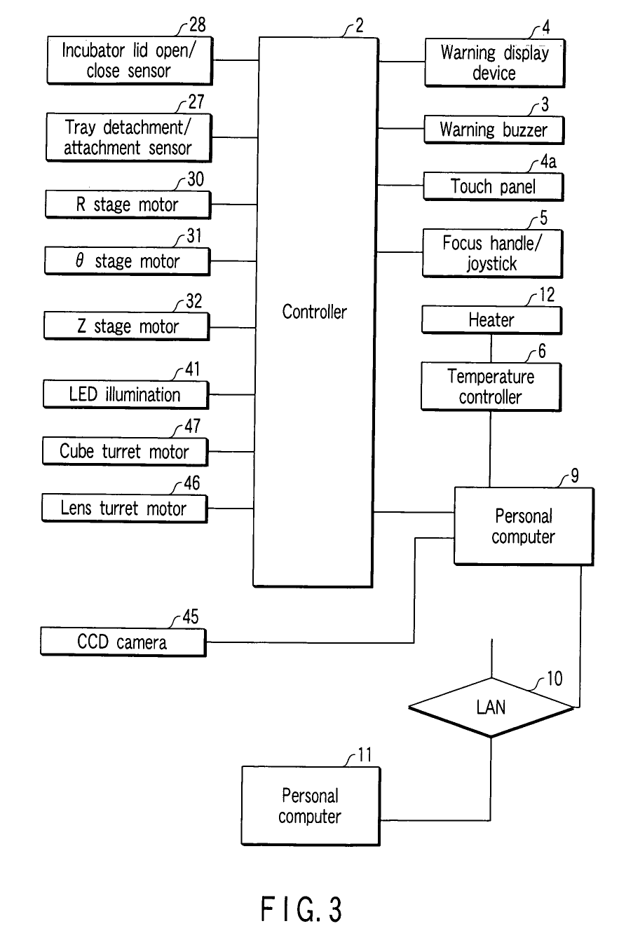

FIG. 3 is a block diagram showing some of the units shown in FIGS. 1 and 2, which can controlled. The units shown in FIG. 3 are connected to the controller 2 so that they may be controlled by the user interface of the computer 9. The CCD camera 45, which is a high-sensitivity type using a cooling CCD, is directly connected to the computer 9. The heater 12 is connected to the computer 9 by the temperature controller 6. Nonetheless, the heater 12 may be controlled by the controller 2 if the controller 2 can perform the function of the temperature controller 6. The CCD camera 45 may be connected to the controller 2, and is controlled by the controller 2, or by the computer 9 through to the controller 2.

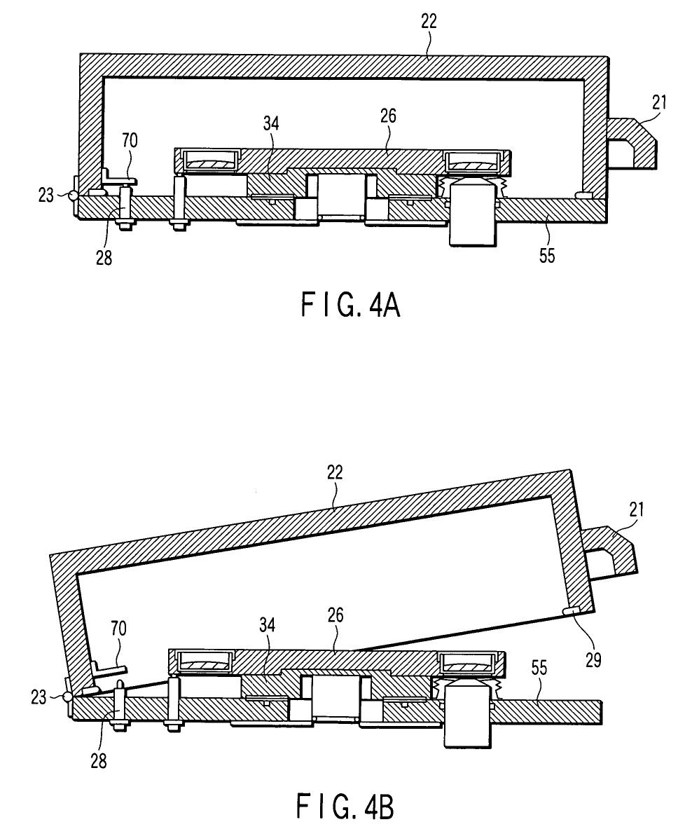

FIG. 4A depicts the incubator chamber 20 in closed state. FIG. 4B shows the incubator chamber 20 opened, so that the culture medium may be replaced by a new one. While the chamber 20 remains closed as illustrated in FIG. 4A, the operator may lift the knob 21. Then, the lid 22 is opened, rotating around the hinge 23 used as a shaft, as is illustrated in FIG. 4B. As the lid 22 is so opened, a member 70 moves away from the lid open/close sensor 28. The sensor 28 is thereby actuated. The sensor 28 generates a signal indicating that the lid 22 has been opened. This signal is supplied to the computer 9 through the controller 2. The user interface of the computer 9 displays an image of the lid 22 thus opened. The lid open/close sensor 28 is of a push-button type, as seen from FIGS. 4A and 4B. The sensor 28 may be replaced by a sensor of any other type that can detect whether the lid 22 is opened or closed.

A rubber layer 29 is laid on that entire contact surface of the lid 22, which contacts the base 55. The layer 29 improves the sealing to the base 55. The layer 29 is pressed when the lid 22 is closed.

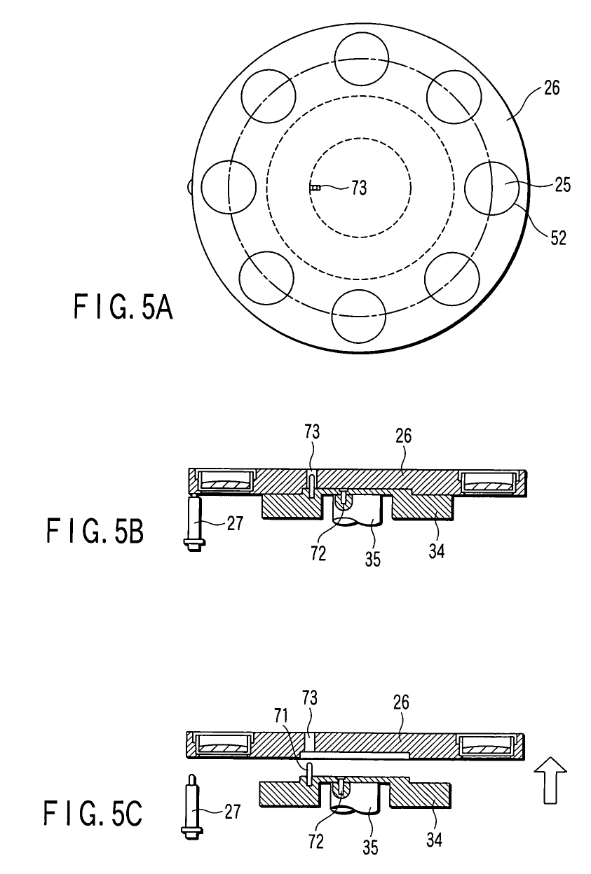

FIGS. 5A, 5B and 5C explain how the tray 26 is removed from the base 34 so that the culture medium may be replaced. FIG. 5A is a top view of the tray 26. The tray 26 has sample-holding holes 52, which are arranged at regular intervals, in a circle around the axis of the θ rotary shaft. FIG. 5B is a side sectional view of the tray 26 and some other components. The θ rotary shaft 35 and the rotary base 34 keep in touch to each other. The rotary base 34 is not removed at the time of observation. The rotary base 34 and the tray 26 can be fastened together and removed from each other. They can be positioned by a positioning pin 71, with the projecting section of the rotary base 34 fitted in the recess made in the tray 26. The tray 26 has an elongate hole 73. The pin 71 is inserted in the elongate hole 73, allowing the tray 26 to move a little in the radial direction, and prohibiting the tray 26 from rotating around the θ rotary shaft. FIG. 5C shows the tray 26 moved upwards from the rotary base 34. When the tray 26 is removed from the base 34, the tray attachment/detachment sensor 27 operates and generates a signal indicating that the tray 26 has been removed. This signal is supplied to the computer 9 through the controller 2. The user interface of the computer 9 displays an image showing the removal of the tray 26.

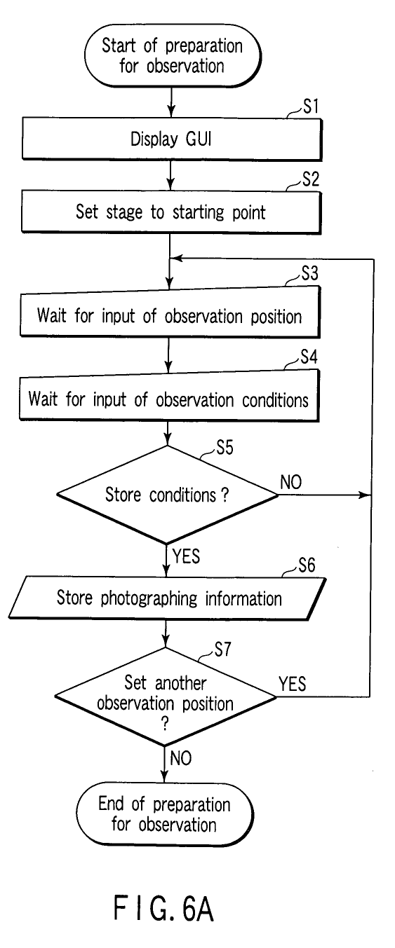

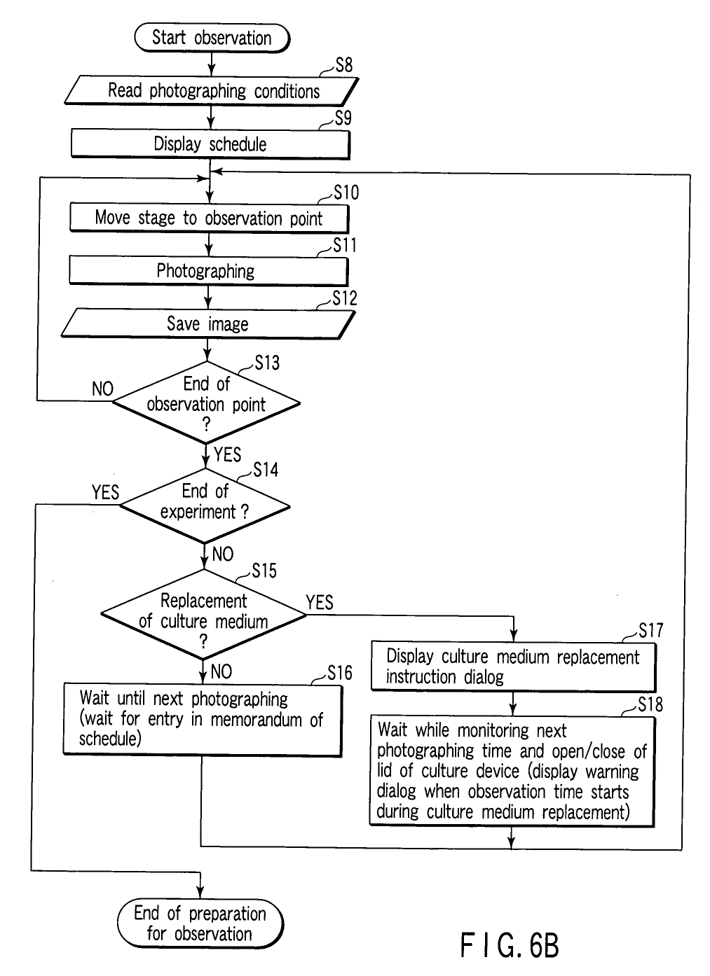

FIGS. 6A and 6B are flowcharts of the computer program which causes the computer 9 to control the culture microscope. The computer program includes an observation preparation program shown in FIG. 6A and an observation start program shown in FIG. 6B. The observation preparation program sets observation conditions. The observation start program includes the program for the time-lapse observation.

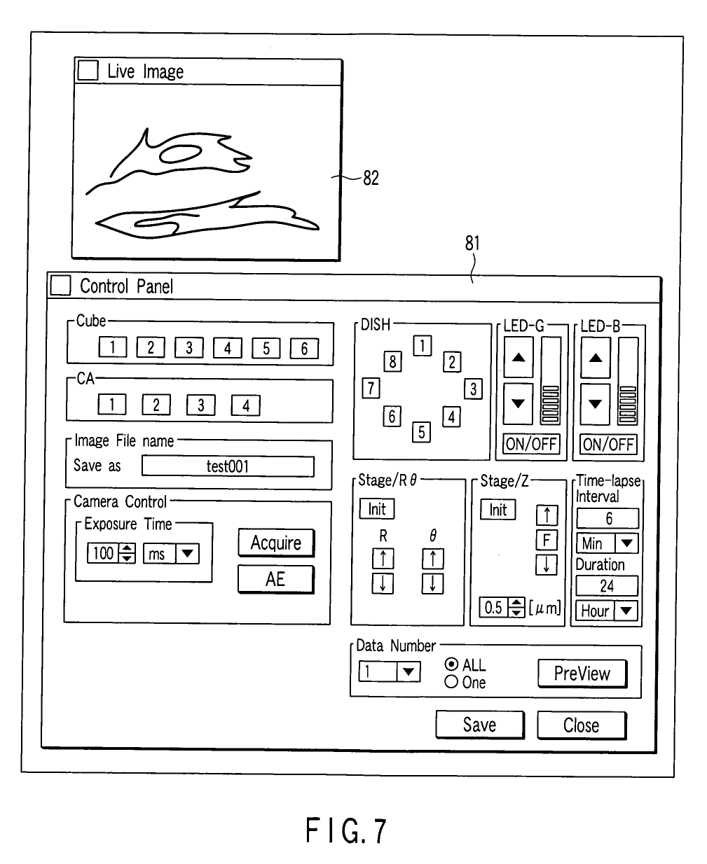

The computer 9 executes the observation preparation program shown in FIG. 6A. Then, a live image window 82 and a control panel GUI 81 are displayed at step S1, in the computer screen as shown in FIG. 7. An image generated by the CCD camera 45 is displayed in the live image window 82 in real time. Seeing the image, the operator may input observation conditions and others. At step S2, an input indicating whether an original position of the stage should be set is waited for. The operator may use a mouse, clicking the Init buttons provided in Stage/Rθ and Stage/Z of the control panel GUI 81. Then, the original position of the stage is set. The computer 9 calculates a distance from the original position to the next observation position, using the original position as reference. The original position is not set when the power supply to the apparatus has just started. The stage is inevitably displaced. It is therefore necessary to set the original position. If the original position has been already set, the step S2 need not be performed.

At step S3, an input of an observation position is waited for. The button number provided in the DISH of the control panel GUI 81 corresponds to one of the sample containers held in the tray 26. Hence, the operator can select any sample container to be observed. After selecting a sample container, the operator pushes an arrow buttons provided in Stage/RO and Stage/Z until the operator finds a cell in the sample container in the image displayed in the live image window 82, thus determining the position of the cell. Stage/X-Y may be displayed in the control panel GUI 81, instead of Stage/Rθ, and the arrow buttons may be arranged in rows and columns. If this is the case, the vertical direction and the horizontal direction correspond to the vertical axis of Y and horizontal direction of X of the live image window 82, respectively.

At step S4, the input of photographing conditions is waited for. LED-G for green light or LED-B for blue light, provided in the control panel GUI 81, is selected for use, and brightness desired for the LED illumination 41 is determined. The fluorescent cube corresponding to a numbered button is selected in Cube, and a magnification-changing lens corresponding to a numbered button is selected in Lens. Further, photographing conditions of the camera, e.g., exposure time of the CCD camera, performing or non-performing of AE, are determined in Camera Control. A file name of saved image after photograph is determined in Image File Name. Interval of the time-lapse is determined in Time-lapse. All parameters required as observation conditions, such as an experiment period, are set. The interval of the time-lapse is the sum of the motorized-stage moving time for photographing the sample at multiple points for the first time, the photographing time, the control time, and the standby time immediately before photographing the sample at the multiple points for the second time, which must be spent on the multipoint time-lapse. This counting is also for photographing the sample at one point only. The interval time is input to the control panel GUI 81. Instead of the interval of the time-lapse, the standby time may be input to the panel GUI 81.

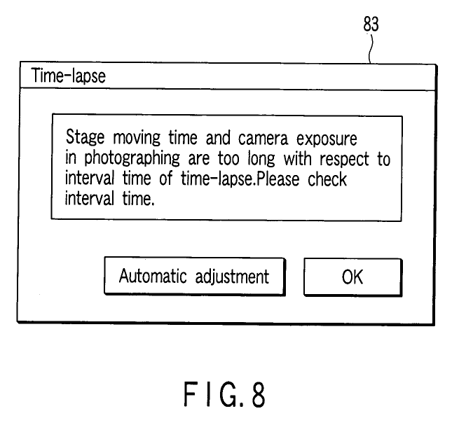

At step S5, it is determined whether the conditions set at the steps S3 and S4 should be stored. If YES, a save button on the control panel GUI 81 is clicked. Conditions are saved as observation data identified by the number in Data Number. When the operator clicks a PreView button of Data Number, one or all of observation data items indicated by the number are executed. It is therefore possible to confirm whether the observation position and conditions are correct. In particular, when the sum of the moving time of the stage and the exposure time of the camera is longer than the interval of the time-lapse, the time-lapse cannot be performed. In this case, a caution dialog 83 is displayed as shown in FIG. 8, prompting the operator to reset the interval time of the time-lapse or to change the number of observation positions. The operator may click an automatic adjustment button. If the operator clicks this button, a time slightly longer than the sum of the movement time of the stage and the exposure time of the camera is automatically calculated and is set as interval of the time-lapse. Alternatively, the standby period may be set to 0 in order to repeat the photographing continuously.

If the conditions are not stored at step S5, the steps S3 and S4 are repeated to determine conditions. If the conditions are stored, they are stored as data in a storage device, such as a hard disk, at step S6.

At step S7, it is determined whether another observation position should be set. If YES, steps S3 and S4 are repeated at the control panel GUI 81 to determine the conditions. When other observation positions are set, the multipoint time-lapse is carried out. Instead of the other observation positions, other photographing conditions such as exposure time of the camera, brightness of the LED illumination, magnification, change of a fluorescent cube and the like may be set. When no other observation position is set, the close button is clicked to close the control panel GUI 81. This terminates the operation.

When the observation is thoroughly prepared, the observation start program shown in FIG. 6B is started. At this time, the data stored at the step S6 is first read from the storage device at a step S8.

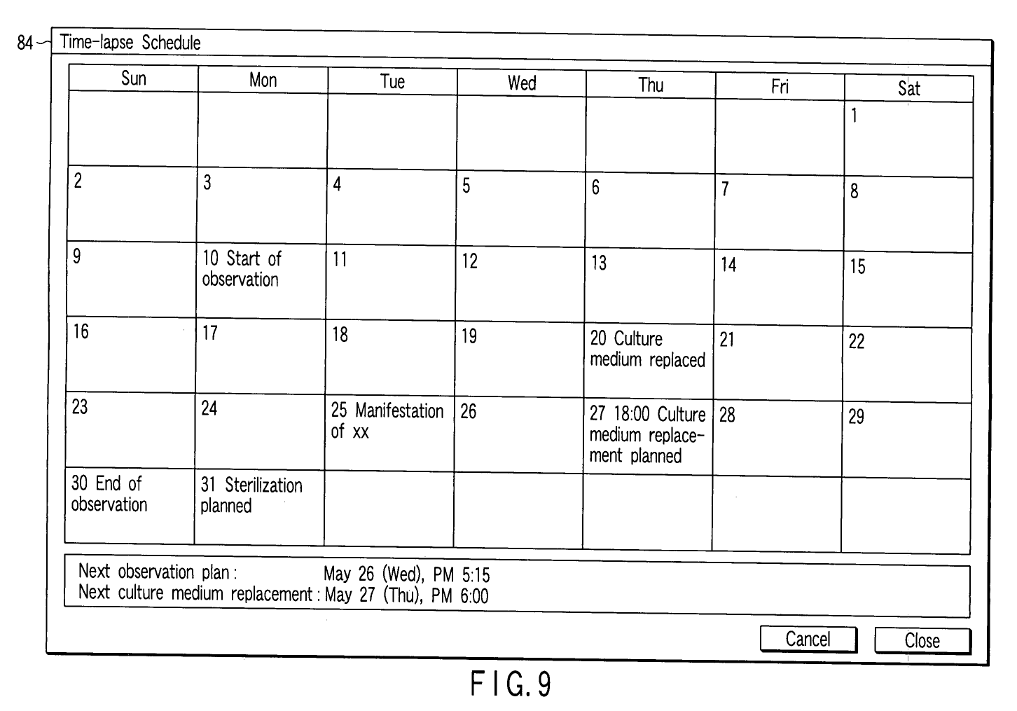

At step S9, a time-lapse schedule window 84 shown in FIG. 9 is displayed in the computer screen. In the time-lapse schedule window 84, a time schedule is displayed as a monthly based calendar. The time and date determined from the conditions set by the observation preparation program shown in FIG. 6A can be displayed in the time-lapse schedule window 84. The conditions or schedule of the multipoint time-lapse, created by the observation preparation program, are thereby made comprehensible. From the conditions displayed in the time-lapse schedule window 84, only the necessary conditions, e.g., a fluorescent cube used at each observation position or the brightness of the LED illumination among the conditions created by the observation preparation program, may be selected and displayed. This renders the displayed data more recognizable. Conditions can be changed by clicking the items displayed in the GUI of the time-lapse schedule window 84. The operator may click, for example, the fluorescent cube. Then, a fluorescent-cube setting dialog, not shown, or an equivalent of the control panel GUI 81 shown in the observation preparation program is displayed, and the fluorescent cube can be reset and saved. Further, an observation position selected for the multipoint time-lapse can be deleted, the order of observation can be changed, or the interval of the multipoint time-lapse can be changed. The observation-end time and date can be calculated from, for example, the observation start time and date and the experiment time for the time-lapse. Hence, it is easy to know when the experiment of time-lapse will end. It is possible to write or note in the time-lapse schedule window 84. The operator can input, if necessary, time and date planned for the replacement of culture medium, or a memorandum concerning the placement of the medium, during the standby time of the time-lapse. The time and date planned for the replacement of culture medium may be automatically calculated from the recorded information of the past. The time-lapse schedule window 84 may be displayed, while the conditions are being set by using the observation preparation program. The next planned observation time and date or the time and date for replacing the culture medium next may be displayed. In this case, the operator can readily confirm the operation timing. When the planned time is reached, the time and date displayed are updated to the next ones.

At step S10, the stages R, θ, and Z are moved to an observation position for a first observation point, in accordance with the photographing condition and the observation-position information read at the step S8.

At step S11, the photographing is started under the photographing condition and the observation position information, both read at step S8. An image is thereby acquired.

At step S12, a serial number is assigned to the file name that specifies the image acquired. The image is saved with avoiding overlapping file names.

At step S13, it is determined whether there are any other observation points. If the next observation point exists, NO is selected at step S13. The control returns to step S10. The stage is moved the next observation point. The sample is then photographed at this observation point, acquiring an image. The image is saved in a file. The sequence of steps S10 to S13 is repeated until the sample is photographed at all observation points.

If no other observation points for the multipoint time-lapse described in the observation preparation program (FIG. 6A) exists, YES is selected at step S13. The control goes to step S14. At step S14, it is determined whether the experiment is terminated. If YES, or if the time and data of the time-lapse have been reached, the observation is terminated. If NO at step S14, the control proceeds to step S15.

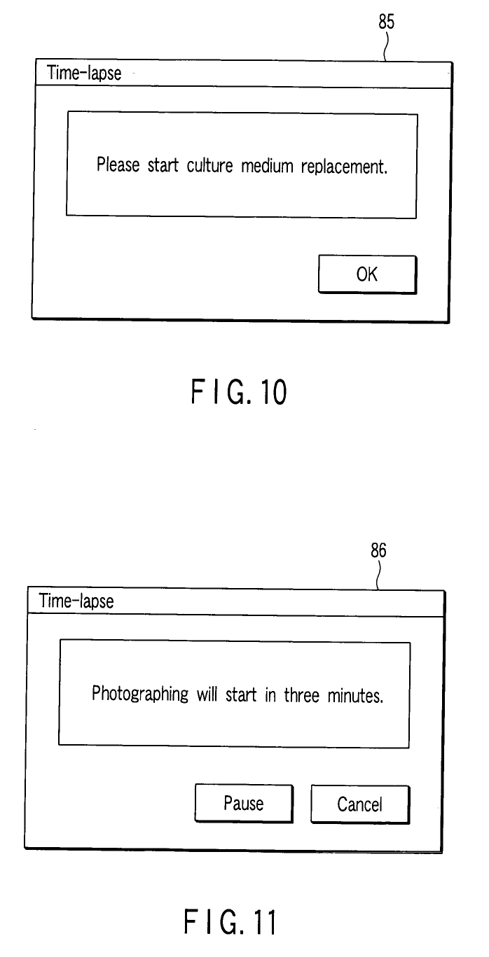

At step S15, it is determined whether the replacement of culture medium is scheduled. If YES at step S15, the control advances to step S17. At step S17, a dialog 85 is displayed as shown in FIG. 10 to prompt the operator to replace the culture medium with a new one.

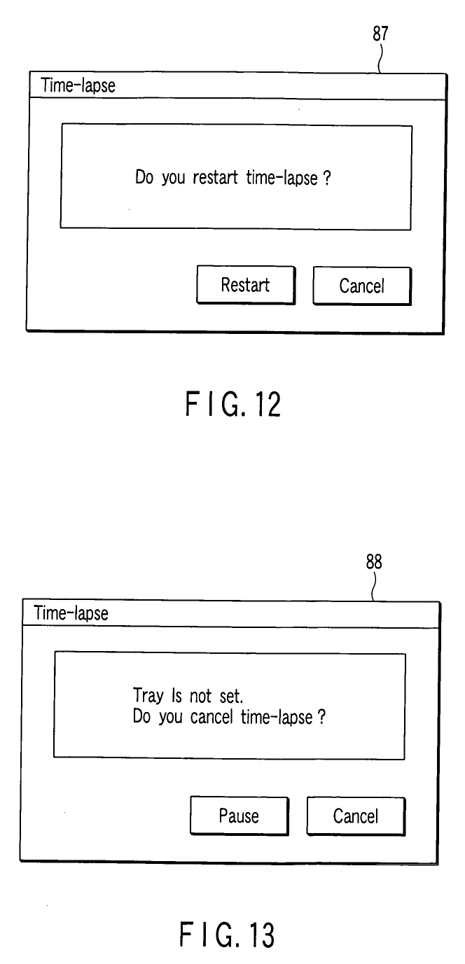

The control then goes to step S18. At step S18, the next photographing time is monitored. At Step 18, too, the output signals of the lid open/close sensor 28 and tray attachment/detachment sensor 27 are monitored to determine whether the lid 22 of the incubator chamber 20 has been opened and whether the tray 26 has been removed from the rotary base 34. To replace the culture medium with a new one, the operator opens the lid 22 and removes the tray 26 from the base 34. Then, the operator may replace the culture medium with a new one on the outside of the apparatus. Then, the sample must be returned to the incubator chamber 20 before the next photographing time comes. During the standby period, a dialog 86 is displayed as illustrated in FIG. 11 to inform how much time is left until the next photographing. Reading the dialog 86, the operator may hurry to finish replacing the medium and inserting the sample back into the incubator chamber 20. The dialog 86 has a pause button. The operator may click the pause button to keep the stage in standby mode even at the time of starting photographing. A restart dialog 87 shown in FIG. 12 is displayed while the stage remains in the standby mode. When it becomes possible to start the time-lapse again, the operator may click a restart button. To terminate the experiment, the operator only needs to click a cancel button.

When the tray 26 remains removed or the lid 22 remains opened at the time of starting photographing, a cancel dialog 88 shown in FIG. 13 is displayed. If nothing is input for a predetermined time, it is determined that an error has occurs. In this case, the experiment is terminated.

If NO at step S15, or if the culture medium is not planned to be replaced, the control advances to step S16. At step S16, the culture microscope is in the standby mode. Thus, the stage is not moved. The sample is not photographed. The operator keeps just waiting. The time-lapse schedule window 84 is displayed, nonetheless. The operator can therefore write a memo or the like in the time schedule, if necessary.

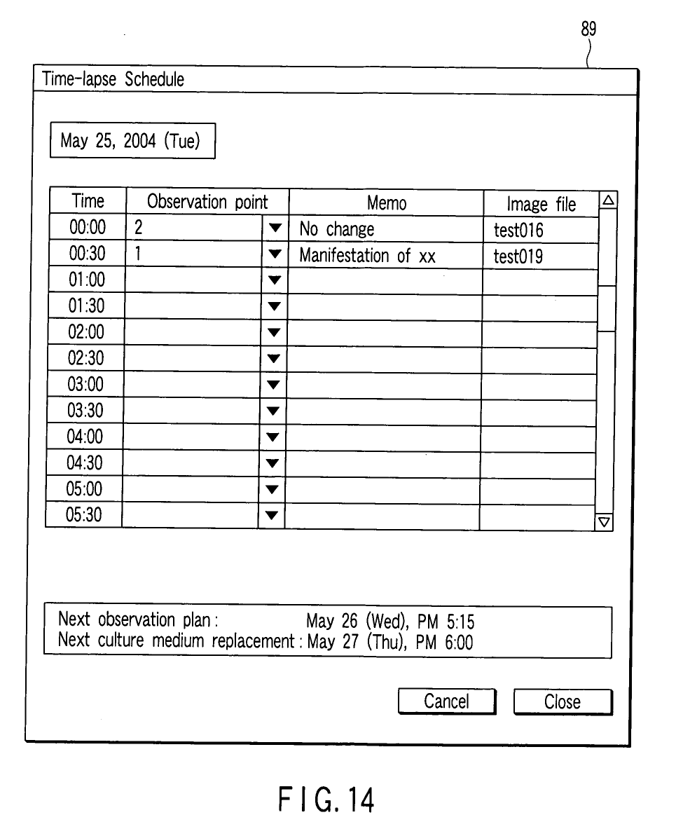

The time schedule shown in FIG. 9 is a monthly based calendar. Instead, a time-lapse schedule window 89 that shows a daily base time schedule may be displayed as illustrated in FIG. 14. In this case, when the operator clicks any date in the time-lapse schedule window 84, the schedule window 89 of FIG. 14 is displayed. The photographing time and date based on the time-lapse of the specified date are displayed as a time schedule which is for planned observation or culture medium replacement, for example. A file name is displayed to identify the image data acquired by photographing the sample. The operator can input a brief comment on the image data and then save the data. If the operator may click the file name, the image represented by the data saved will be displayed. Seeing the image displayed, the operator can confirm whether the sample has been appropriately photographed.

When the next photographing time for the multipoint time-lapse comes at step S16 or step S18, the control returns to step S10. At step S10, the stage is moved to the observation point, and the photographing is repeated.

The displayed GUI or the operation can be controlled at a remote site at a distance from the culture microscope by the personal computer 11 via the LAN 10 shown in FIG. 1.

In the first embodiment, the time that the operator can spend to open the incubator chamber 20, remove the tray 26 and replace the culture medium, during the standby period in the automatic observation based on the multipoint time-lapse, is displayed. This prompts the operator to finish replacing the medium within the time displayed. Further, the time-lapse schedule window 84 is displayed to show the operator the entire multipoint time-lapse planned, the next operation and the like. Since the time-lapse schedule window 84 is displayed, the operator can easily change various conditions of the multipoint time-lapse set in the observation preparation program (FIG. 6A). The plan of the multipoint time-lapse once set and of the various conditions set for the multipoint time-lapse can therefore be changed easily.

A second embodiment of the present invention will be described with reference to the accompanying drawings.

The apparatus configuration, the program for preparing observation and the program for starting observation are the same as those in the first embodiment. Therefore, they will not be described in detail.

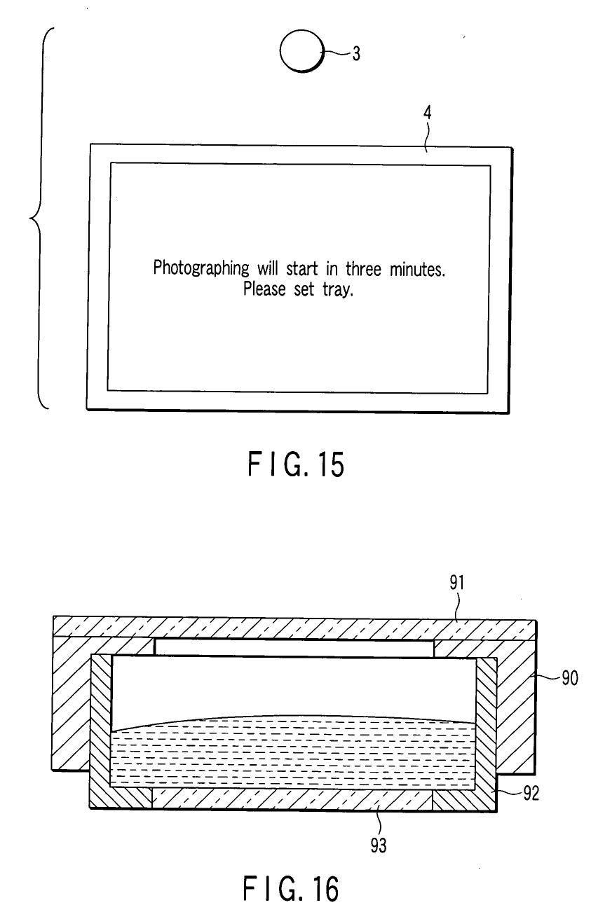

FIG. 15 depicts the alarm buzzer 3 and the alarm display 4. The alarm display 4 can display the information as displayed in the monitor of the computer 9 and/or as in the form of characters and the like. The display 4 can display, for example, the dialog 85 shown in FIG. 10 for prompting the operator to replace the culture medium that has been explained in conjunction with step S17 in FIG. 6B. The dialog 85 helps the operator to determine whether the culture medium is readily replaced or not, because the replacing of the culture medium is often carried out in the vicinity of the culture microscope main body 1.

Moreover, the alarm buzzer 3 may be driven to generate an alarm when displaying the cancel dialog 88 shown in FIG. 13 is displayed. Hearing the alarm and seeing the dialog 88, the operator can early know that the photographing should be cancelled.

As described above, according to the second embodiment, it is possible to early impart information to an operator.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A culture microscope comprising:
culturing means for controlling a culture environment in which a cell is cultured;
imaging means for photographing the cell; and
controlling means for controlling time-lapse photographing performed by the imaging means.

2. The culture microscope according to claim 1, further comprising displaying means for displaying settings or states of the imaging means.

3. The culture microscope according to claim 2, wherein the displaying means displays a time remaining until the next photographing during a standby period.

4. The culture microscope according to claim 1, further comprising displaying means for displaying a photographing time and date set in the controlling means as a time schedule.

5. The culture microscope according to claim 4, wherein the time schedule is writable.

6. The culture microscope according to claim 1, further comprising detecting means for detecting whether the apparatus is in an imaging enabled state, and displaying means for displaying settings and states of the imaging means.

7. The culture microscope according to claim 6, further comprising alarm means for issuing an alarm when the apparatus is in an imaging disabled state at an imaging start timing.

8. The culture microscope according to claim 7, wherein the alarm means comprises an alarm display device.

9. The culture microscope according to claim 7, wherein the alarm means comprises an alarm buzzer.

10. The culture microscope according to claim 1, wherein the imaging means has an objective lens, an imaging device, and a magnification-changing lens which is inserted into and removed from an optical path extending from the objective lens to the imaging device.

11. The culture microscope according to claim 1, wherein the culture microscope can be operated by a computer at a remote site.

12. A computer program which causes a computer to control the culture microscope defined in claim 4, the computer program having the time schedule.

13. A culture microscope comprising:
culturing means for maintaining a culture environment in which a cell is cultured;
imaging means for photographing a cell image; and
controlling means for controlling a time-lapse photographing which is performed by the imaging means.

14. The culture microscope according to claim 13, further comprising displaying means for displaying settings or states of the imaging means.

15. The culture microscope according to claim 14, wherein the displaying means displays a time remaining until the next photographing during a standby period.

16. The culture microscope according to claim 13, further comprising displaying means for displaying a photographing time and date set in the controlling means as a time schedule.

17. The culture microscope according to claim 16, wherein the time schedule is writable.

18. The culture microscope according to claim 13, further comprising detecting means for detecting whether the apparatus is in a photographing enabled state and displaying means for displaying settings or states of the imaging means.

19. The culture microscope according to claim 18, further comprising alarm means for issuing an alarm when the apparatus is in an imaging disabled state at an imaging start timing.

20. The culture microscope according to claim 19, wherein the alarm means comprises an alarm display device.

21. The culture microscope according to claim 19, wherein the alarm means comprises an alarm buzzer.

22. The culture microscope according to claim 13, wherein the imaging means has an objective lens, an imaging device, and a magnification-changing lens which is inserted into and removed from an optical path extending from the objective lens to the imaging device.

23. The culture microscope according to claim 13, wherein the culture microscope can be operated by a computer at a remote site.

24. A computer program which causes a computer to control the culture microscope defined in claim 16, the computer program having the time schedule.

25. A culture microscope comprising:
an incubator chamber which controls a culture environment in which a cell is cultured;
an imaging optical system which photographs the cell; and
a controller which controls a time-lapse photographing which is performed by the imaging optical system.

26. The culture microscope according to claim 25, further comprising a display device which displays settings or states of the imaging optical system.

27. The culture microscope according to claim 26, wherein the displaying device displays a time remaining until the next photographing during a standby period.

28. The culture microscope according to claim 25, further comprising a display device which displays a photographing time and date set in the controller as a time schedule.

29. The culture microscope according to claim 28, wherein the time schedule is writable.

30. The culture microscope according to claim 25, further comprising a sensor which detects whether the apparatus is in an imaging enabled state and a display device which displays settings or states of the imaging optical system.

31. The culture microscope according to claim 30, further comprising an alarm device which issues an alarm when the apparatus is in an imaging disabled state at an imaging start timing.

32. The culture microscope according to claim 31, wherein the alarm device comprises an alarm display device.

33. The culture microscope according to claim 31, wherein the alarm device comprises an alarm buzzer.

34. The culture microscope according to claim 25, wherein the imaging optical system has an objective lens, an imaging device, and a magnification-changing lens which is inserted into and removed from an optical path extending from the objective lens to the imaging device.

35. The culture microscope according to claim 25, wherein the culture microscope can be operated by a computer at a remote site.

36. A computer program which causes a computer to control the culture microscope defined in claim 28, the computer program having the time schedule.

37. A culture microscope comprising:
an incubator chamber which maintains a culture environment in which a cell is cultured;
an imaging optical system which photographs the cell; and
a controller which controls a time-lapse photographing which is performed by the imaging optical system.

38. The culture microscope according to claim 37, further comprising a display device which displays settings or states of the imaging optical system.

39. The culture microscope according to claim 38, wherein he displaying device displays a time remaining until the next photographing during a standby period.

40. The culture microscope according to claim 37, further comprising a display device which displays a photographing time and date set in the controller as a time schedule.

41. The culture microscope according to claim 40, wherein the time schedule is writable.

42. The culture microscope according to claim 37, further comprising a sensor which detects whether the apparatus is in an imaging enabled state and a display device which displays settings or states of the imaging optical system.

43. The culture microscope according to claim 42, further comprising an alarm device which issues an alarm when the apparatus is in an imaging disabled state at an imaging start timing.

44. The culture microscope according to claim 43, wherein the alarm device comprises an alarm display device.

45. The culture microscope according to claim 43, wherein the alarm device comprises an alarm buzzer.

46. The culture microscope according to claim 37, wherein the imaging optical system has an objective lens, an imaging device, and a magnification-changing lens which is inserted into and removed from an optical path extending from the objective lens to the imaging device.

47. The culture microscope according to claim 37, wherein the culture microscope can be operated by a computer at a remote site.

48. A computer program which causes a computer to control the culture microscope defined in claim 40, the computer program having the time schedule.
